(19) United States
(12) Patent Application Publication
(10) Pub. No.: US 2022/0145388 A1
(43) Pub. Date: May 12, 2022

(54) METHODS OF IDENTIFYING SNPS CORRELATING WITH ELITE ATHLETIC PERFORMANCE

(71) Applicants: Qatar Foundation for Education, Science and Community Development, Doha (QA); Qatar University, Doha (QA); Anti-Doping Lab Qatar, Doha (QA)

(72) Inventors: Mohamed Elrayess, Doha (QA); Fatima Al-Khelaifi, Doha (QA)

(21) Appl. No.: 17/476,902

(22) Filed: Sep. 16, 2021

Related U.S. Application Data
(60) Provisional application No. 63/079,040, filed on Sep. 16, 2020.

(51) Int. Cl. C12Q 1/6876

(52) U.S. Cl. CPC C12Q 1/6876; C12Q 2600/156; C12Q 2600/124

(57) ABSTRACT

The present disclosure, in part, relates to novel, methods of assessing an individual's genetic predisposition to elite athleticism. The present disclosure includes methods for identifying individuals with SNPs that are in linkage disequilibrium with SNP rs1052373 on the MYBPC3 gene. The present disclosure also includes next-generation doping tests.

### CROSS-REFERENCE TO RELATED APPLICATIONS

This application claims the benefit of and priority to U.S. Provisional Patent Application No. 63/079,040 filed on Sep. 16, 2020, the entire contents of this application are incorporated herein by reference in its entirety for all purposes.

### BACKGROUND

Elite athletic performance is a multi-factorial trait with input from both genetic and environmental factors. Historically, the superior performance of elite athletes has been considered a result of special talent shaped by intensive training. The talent is now believed to be a product of additive genetic components predisposing the athlete to endurance, speed, strength, flexibility and coordination trainability under the control of strong environmental cues including exercise and nutrition. In this model, the genetic predisposition together with ability to respond to training are the keys to the superior physical performance of elite athletes.

Sports can be classified according to the type and intensity of the exercise required to perform during competition. For example, the percentage of maximal oxygen uptake (VO₂ₘₐₓ) reflects the maximal cardiac output, the oxygen transport capacity, and the blood volume. Accordingly, sports can be divided into events with low, moderate and high aerobic (dynamic) components. Similarly, the percent of maximal voluntary contraction (MVC), which reflects the greatest amount of tension a muscle can generate and hold, is used to classify sports into sporting disciplines with low, moderate and high power components.

Classical twin and family genetic studies have suggested that VO₂ₘₐₓ is up to 94% inherited. Genome-wide association studies (GWAS) in athletes versus non-athletes have uncovered many new loci in association with VO₂ₘₐₓ and elite endurance performance. A more recent review of genetic predisposition to elite athletic endurance has highlighted 100 endurance variants. However, despite some initial evidence suggesting identification of genetic variants in GWAS studies, further studies did not replicate/validate these findings hindered by a small sample size and complex phenotype. One of the first GWAS in athletes using K single-nucleotide polymorphisms (SNPs) and subsequent meta-analysis of 45 promising genetic markers in 1520 endurance athletes and 2760 controls has revealed only one statistically significant marker (rs558129 at GALNTL6) associated with endurance status in world class athletes, but not at genome wide level of significance. Therefore, the genetic predisposition to endurance traits remains unclear, largely due to the relatively underpowered elite athletes' cohorts. Recently, a polymorphism in human homeostatic iron regulator protein was found to be associated with elite endurance athlete status and aerobic capacity in Russian athletes.

Metabolomics analysis has presented a novel tool to validate genomics data by providing an intermediate phenotype (metabolites) in association with the identified genetic variants. Pilot metabolomics studies have revealed differences in the metabolic signature of moderate and high endurance elite athletes, such as steroid biosynthesis, fatty acid metabolism, oxidative stress and energy-related molecular pathways. Recently, a study investigating metabolic GWAS of elite athletes showed novel genetically-influenced metabolites associated with athletic performance. These included two novel genetic loci in FOLH1 and VNN1 in association with N-acetyl-aspartyl-glutamate and linoleoyl ethanolamide, respectively, and one novel locus linking genetic variant in SULT2A1 and androstenediol (3alpha, 17alpha) monosulfate in endurance athletes.

### SUMMARY

The present disclosure, in part, relates to a novel method of identifying individuals with a genetic predisposition to elite athletic performance. For example, in embodiments, the genetic predisposition comprises two SNPs in linkage disequilibrium, such as SNPs within the genes MYBPC3 and NR1H3. Generally, the methods disclosed herein relate to identification of individuals with SNPs rs7120118 in gene NR1H3 and rs1052373 in the gene MYBPC3.

Various non-exhaustive, non-limiting aspects according to the present disclosure may be useful alone or in combination with one or more other aspects described herein. Without limiting the foregoing description, in a first non-limiting aspect of the present disclosure, a method of screening elite athletic candidates for endurance sports ability.

In accordance with a second non-limiting aspect of the present disclosure, which may be used in combination with the first aspect, the method comprises identifying the presence of SNP rs1052373 in the MYBPC3 gene.

In accordance with a third non-limiting aspect of the present disclosure, which may be used in combination with each or any of the above-mentioned aspects, the method comprises determining whether the individual or subject with SNP rs1052373 is a GG homozygote.

In accordance with a fourth non-limiting aspect of the present disclosure, which may be used in combination with each or any of the above-mentioned aspects, the method comprises assessing VO₂ₘₐₓ in individuals positive for SNP rs1052373.

In accordance with a fifth non-limiting aspect of the present disclosure, which may be used in combination with each or any of the above-mentioned aspects, the method comprises assessing endurance athletic ability in individuals positive for SNP rs1052373.

In accordance with a sixth non-limiting aspect of the present disclosure, which may be used in combination with each or any of the above-mentioned aspects, the method comprises genotyping individuals and determining whether the individuals have SNPs that are in linkage disequilibrium with SNP rs1052373.

In accordance with a seventh non-limiting aspect of the present disclosure, which may be used in combination with each or any of the above-mentioned aspects, the method comprises identifying whether individuals with SNP rs1052373 are carriers of either of the AA+AG alleles.

In accordance with an eighth non-limiting aspect of the present disclosure, which may be used in combination with each or any of the above-mentioned aspects, the method comprises measuring testosterone levels of individuals with SNP rs1052373.

In accordance with a ninth non-limiting aspect of the present disclosure, which may be used in combination with each or any of the above-mentioned aspects, the method comprises measuring the level of the testosterone precursor, androstenediol (3beta, 17beta) disulfate in individuals with SNP rs1052373.

In accordance with a tenth non-limiting aspect of the present disclosure, which may be used in combination with each or any of the above-mentioned aspects, the embodiment of evaluating the training program of an individual with SNP rs1052373.

In accordance with an eleventh non-limiting aspect of the present disclosure, which may be used in combination with the first aspect, the method comprises identifying the presence of SNP rs7120118 in the NR1H3 gene.

In accordance with a twelfth non-limiting aspect of the present disclosure, which may be used in combination with each or any of the above-mentioned aspects, the method comprises assessing VO₂ₘₐₓ in individuals positive for SNP rs7120118.

In accordance with a thirteenth non-limiting aspect of the present disclosure, which may be used in combination with each or any of the above-mentioned aspects, the method comprises assessing endurance athletic ability in individuals positive for SNP rs7120118.

In accordance with a fourteenth non-limiting aspect of the present disclosure, which may be used in combination with each or any of the above-mentioned aspects, the method comprises measuring testosterone levels of individuals with SNP rs7120118.

In accordance with a fifteenth non-limiting aspect of the present disclosure, which may be used in combination with each or any of the above-mentioned aspects, the method comprises measuring the level of the testosterone precursor, androstenediol (3beta, 17beta) disulfate in individuals with SNP rs7120118.

In accordance with a sixteenth non-limiting aspect of the present disclosure, which may be used in combination with each or any of the above-mentioned aspects, the embodiment of a evaluating the training program of an individual with SNP rs7120118.

In accordance with a seventeenth non-limiting aspect of the present disclosure, which may be used in combination with each or any of the above-mentioned aspects, the method comprises a next-generation doping test.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A depicts GWAS data quality control. Principle component analysis (PCA) shows no difference in the genotype distribution among sport disciplines.

FIG. 1B depicts GWAS data quality control. Principle component analysis (PCA) shows no difference in the genotype distribution between groups (sports with low/moderate vs high aerobic component).

FIG. 1C depicts GWAS data quality control. Manhattan (arrow indicates significant SNPs identified in meta-analysis) plots illustrating GWAS results in association with endurance.

FIG. 1D depicts GWAS data quality control. Quantile-quantile (no evidence of genomic inflation, lambda GC=1.006) plots illustrating GWAS results in association with endurance.

FIG. 2 shows a regional association plot for the region around rs1052373. The colors correspond to different LD thresholds, where LD is computed between the sentinel SNP (lowest p-value, colored in blue) and all SNPs. Shapes of markers correspond to their functionality as described in the legend.

FIG. 3 shows boxplots representing levels of 5alpha-androstan-3alpha and 17alpha-diol disulfate in rs7120118 and rs1052373 genotype groups.

### DETAILED DESCRIPTION

### List of Abbreviations

ACP2 (Acid Phosphatase 2, Lysosomal).

Anti-doping laboratories in Qatar (ADLQ).

False discovery rate (FDR).

Genome variation server (GVS).

Genome-wide association studies (GWAS).

High resolution/accurate mass spectrometer interfaced with a heated electrospray ionization (HESI-II).

Laboratorio Antidoping, Federazione Medico Sportiva Italiana (FMSI).

MAP Kinase Activating Death Domain (MADD).

Maximal oxygen uptake (VO₂max).

Maximal voluntary contraction (MVC).

Minor allele frequency (MAF).

Myosin Binding Protein C, Cardiac (MYBPC3).

Nuclear Receptor Subfamily 1 Group H Member 3 (NR1H3).

Odds Ratio (OR).

Spi-1 (Spi-1 Proto-Oncogene).

Ultra-performance liquid chromatography (UPLC).

### Definitions

As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number.

All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this disclosure and the appended claims, the singular forms “a,” “an” and “the” include plural referents unless the context clearly dictates otherwise. Thus, for example, reference to “a component” or “the component” includes two or more components.

The words “comprise,” “comprises” and “comprising” are to be interpreted inclusively rather than exclusively. Likewise, the terms “include,” “including,” “containing” and “having” should all be construed to be inclusive, unless such a construction is clearly prohibited from the context. Further in this regard, these terms specify the presence of the stated features but not preclude the presence of additional or further features.

Nevertheless, the methods disclosed herein may lack any element that is not specifically disclosed herein. Thus, a disclosure of an embodiment using the term “comprising” is (i) a disclosure of embodiments having the identified components or steps and also additional components or steps, (ii) a disclosure of embodiments “consisting essentially of” the identified components or steps, and (iii) a disclosure of embodiments “consisting of” the identified components or steps. Any embodiment disclosed herein can be combined with any other embodiment disclosed herein.

The term “and/or” used in the context of “X and/or Y” should be interpreted as “X,” or “Y,” or “X and Y.” Similarly, “at least one of X or Y” should be interpreted as “X,” or “Y,” or “X and Y.”

Where used herein, the terms “example” and “such as,” particularly when followed by a listing of terms, are merely exemplary and illustrative and should not be deemed to be exclusive or comprehensive.

A “subject” or “individual” is a mammal, preferably a human.

All percentages expressed herein are by weight of the total weight of the composition unless expressed otherwise. When reference herein is made to the pH, values correspond to pH measured at about 25° C. with standard equipment. “Ambient temperature” or “room temperature” is between about 15° C. and about 25° C., and ambient pressure is about 100 kPa.

The term “mM”, as used herein, refers to a molar concentration unit of an aqueous solution, which is mmol/L. For example, 1.0 mM equals 1.0 mmol/L.

The terms “peptide” or “protein” or “polypeptide” refers to a polymer of amino acid residues covalently linked by peptide bonds. The terms “peptides” or “proteins” or “polypeptides,” used herein, may also refer to a polymer of amino acids where one or more of the amino acids may be a modified residue, such as an artificial amino acid mimetic or a synthetic amino acid residue. The terms “peptide” or “protein” or “polypeptide” are used interchangeably.

The term “segment,” when used in reference to a “peptide” or “protein” or “polypeptide,” refers to the entire sequence and in addition, optionally, refers to a portion of that “peptide” or “protein” or “polypeptide” that is at least one or more of the amino acids, but less than the entire sequence of the “peptide” or “protein” or “polypeptide.”

The terms “nucleic acid” or “genetic material” or “polynucleotide” refer to “deoxyribonucleic acid” (DNA) or “ribonucleic acid” (RNA) and polymers thereof, in either single- or double-stranded form.

The terms “treatment” and “treat” include both prophylactic or preventive treatment (that prevent and/or slow the development of a targeted pathologic condition, infection, disorder, or disease) and curative, therapeutic or disease-modifying treatment, including therapeutic measures that cure, slow down, lessen symptoms of, and/or halt progression of a diagnosed pathologic condition, infection, disorder, or disease. The terms “treatment” and “treat” do not necessarily imply that a subject is treated until total recovery. The terms “treatment” and “treat” are also intended to include the potentiation or otherwise enhancement of one or more primary prophylactic or therapeutic measures. As non-limiting examples, a treatment can be performed by a doctor, a healthcare professional, a veterinarian, a veterinarian professional, or another human.

The terms “substantially no,” “essentially free” or “substantially free” as used in reference to a particular component means that any of the component present constitutes no more than about 3.0% by weight, such as no more than about 2.0% by weight, no more than about 1.0% by weight, preferably no more than about 0.5% by weight or, more preferably, no more than about 0.1% by weight.

### DETAILED DESCRIPTION

Single Nucleotide Polymorphisms (SNPs) are germline substitutions of a single nucleotide at a specific position in the genome. For example, at a specific base position in the human genome, the G nucleotide may appear in most individuals, but in a minority of individuals, the position is occupied by an A. This means that there is a SNP at this specific position, and the two possible nucleotide variations—G or A—are said to be the alleles for this specific position. More than 335 million SNPs have been found across humans from multiple populations. A typical genome differs from the reference human genome at 4 to 5 million sites, most of which (more than 99.9%) consist of SNPs and short insertions/deletions. There are variations between human populations, so a SNP allele that is common in one geographical or ethnic group may be much rarer in another.

SNPs pinpoint differences in our susceptibility to a wide range of diseases (e.g. sickle-cell anemia, β-thalassemia and cystic fibrosis). The severity of illness and the way the body responds to treatments are also manifestations of genetic variations caused by SNPs. For example, a single-base mutation in the APOE (apolipoprotein E) gene is associated with a lower risk for Alzheimer's disease.

Disclosed embodiments utilize the occurrence of SNPs to “screen” or identify individuals with a genetic predisposition, for example a genetic predisposition to elite athletic performance. For example, the presence of specific SNPs and their relationship can increase the potential for an individual to be capable of elite athletic performance. In embodiments, the elite athletic performance can comprise endurance sports performance, strength sports performance, speed sports performance, and combinations thereof. For example, disclosed embodiments can comprise identification of an individual with a genetic predisposition to elite long-distance running performance, for example elite marathon performance. Further disclosed embodiments can comprise identification of an individual with a genetic predisposition to elite sprint performance, such as elite 100m sprint performance, or elite 50m swimming performance.

For example, in embodiments, disclosed methods comprise identification of SNPs within, for example, genes such as MYBPC3 and NR1H3. In embodiments, the SNPs identified can comprise rs7120118 in gene NR1H3 and rs1052373 in the gene MYBPC3. In embodiments, identification of these SNPs can aid in identifying an individual with a genetic predisposition to elite athletic performance.

In further embodiments, the method comprises identifying the presence of SNP rs1052373 in the MYBPC3 gene. Additional embodiments comprise determining whether the individual or subject with SNP rs1052373 is a GG homozygote. In embodiments, identification of these SNPs and alleles can aid in identifying an individual with a genetic predisposition to elite athletic performance.

In additional embodiments, disclosed methods comprise assessing VO₂ₘₐₓ in individuals positive for SNP rs1052373. In additional embodiments, disclosed methods comprise assessing endurance athletic ability in individuals positive for SNP rs1052373.

In embodiments, this genetic predisposition is determined by the presence of at least two SNPs in linkage disequilibrium (LD). In population genetics, LD is the non-random association of alleles at different loci in a given population. Loci are said to be in linkage disequilibrium when the frequency of association of their different alleles is higher or lower than what would be expected if the loci were independent and associated randomly. Linkage disequilibrium is influenced by many factors, including selection, the rate of genetic recombination, mutation rate, genetic drift, the system of mating, population structure, and genetic linkage. As a result, the pattern of linkage disequilibrium in a genome is a powerful signal of the population genetic processes that are structuring it.

Further disclosed methods comprise genotyping individuals and determining whether the individuals have SNPs that are in linkage disequilibrium with SNP rs1052373. In additional embodiments, disclosed methods comprise identifying whether individuals with SNP rs1052373 are carriers of either of the AA+AG alleles. In embodiments, identification of these SNPs and alleles can aid in identifying an individual with a genetic predisposition to elite athletic performance.

Further embodiments comprise measuring testosterone levels of individuals with SNP rs1052373.

Further embodiments comprise measuring the level of the testosterone precursor, androstenediol (3beta, 17beta) disulfate in individuals with SNP rs1052373.

Further embodiments comprise evaluating the training program of an individual with SNP rs1052373.

Further embodiments comprise identifying the presence of SNP rs7120118 in the NR1H3 gene.

Further embodiments comprise assessing VO₂ₘₐₓ in individuals positive for SNP rs7120118. In embodiments comprising assessing VO₂ₘₐₓ in individuals positive for SNP rs7120118, further embodiments comprise assessing endurance athletic ability in individuals positive for SNP rs7120118.

Further embodiments comprise measuring testosterone levels of individuals with SNP rs7120118.

Further embodiments comprise measuring the level of the testosterone precursor, androstenediol (3beta, 17beta) disulfate in individuals with SNP rs7120118.

Further embodiments comprise evaluating the training program of an individual with SNP rs7120118.

Further embodiments comprise a next-generation doping test. For example, in disclosed embodiments, a doping test can comprise identification of SNPs within, for example, genes such as MYBPC3 and NR1H3. In embodiments, the SNPs identified can comprise rs7120118 in gene NR1H3 and rs1052373 in the gene MYBPC3. In embodiments, methods of testing for doping can comprise measuring testosterone levels of individuals with SNP rs1052373, measuring the level of the testosterone precursor, androstenediol (3beta, 17beta) disulfate in individuals with SNP rs1052373, and evaluating the training program of an individual with SNP rs1052373.

In embodiments, methods of testing for doping can comprise measuring testosterone levels of individuals measuring testosterone levels of individuals with SNP rs7120118, measuring the level of the testosterone precursor, androstenediol (3beta, 17beta) disulfate in individuals with SNP rs7120118, and evaluating the training program of an individual with SNP rs7120118.

### EXAMPLES

### Example 1

Background: The genetic predisposition to elite athletic performance has been a controversial subject due to the underpowered studies and the small effect size of identified genetic variants. The aims of this study were to investigate the association of common single-nucleotide polymorphisms (SNPs) with endurance athlete status in a large cohort of elite European athletes using GWAS approach, followed by replication studies in Russian and Japanese elite athletes and functional validation using metabolomics analysis.

Results: The association of 476,728 SNPs of IIlumina DrugCore Gene chip and endurance athlete status was investigated in 796 European international-level athletes (645 males, 151 females) by comparing allelic frequencies between athletes specialized in sports with high (n=662) and low/moderate (n=134) aerobic component. Replication of results was performed by comparing the frequencies of the most significant SNPs between 242 and 168 elite Russian high and low/moderate aerobic athletes, respectively, and between 60 elite Japanese endurance athletes and 406 controls. A meta-analysis has identified rs1052373 (GG homozygotes) in Myosin Binding Protein (MYBPC3; implicated in cardiac hypertrophic myopathy) gene to be associated with endurance athlete status (P=1.43E-08, odd ratio 2.2). Homozygotes carriers of rs1052373 G allele in Russian athletes had significantly greater VO₂ₘₐₓ than carriers of the AA+AG (P=0.005). Subsequent metabolomics analysis revealed several amino acids and lipids associated with rs1052373 G allele (1.82×10⁻⁰⁵) including the testosterone precursor androstenediol (3beta, 17beta) disulfate.

Conclusions: This is the first report of genome-wide significant SNP and related metabolites associated with elite athlete status. Further investigations of the functional relevance of the identified SNPs and metabolites in relation to enhanced athletic performance are warranted.

In this study, we aimed to investigate the association of multiple SNPs and endurance athlete status in a relatively large cohort of European elite athletes specialized in sports with high and low/moderate aerobic component using GWAS approach and replicate our findings in elite Russian and Japanese athletes. We also aimed to perform functional validation using VO₂ₘₐₓ testing and metabolomics analysis by identifying metabolites that are associated with significant endurance-related SNPs.

Results

Genome-Wide Association Study

Athletes from the discovery cohort were classified into different groups of sports following 151 previously published sports classification criteria, as shown in Table 1.

TABLE 1
Classification of GWAS participants (Males: M, Females: F) according to
sports classes. Distribution of elite athletes in various categories based on
sport type-associated peak dynamic (maximal oxygen uptake percentage;
VO₂ₘₐₓ) and peak static (maximal voluntary muscle contraction percentage;
MVC) components achieved during competition.

| | Low/moderate (<70% | | | | | |
|---|---|---|---|---|---|---|
| | VO2max) | | High >70% VO2max) | | | Total |
| High | Wrestling | Skate | Modern Pentathlon (1F) | | | 287 |
| (>50% | and Judo | boarding | Kayaking | Rowing | Biathlon | (71% M) |
| MVC) | (8M) | (2M) | (1F) | (9M/8F) | (2M/1F) | |
| | Weightlifting (14M/7F) | | Boxing | Cycling | Triathlon | |
| | | | (4M/7F) | (157M/49F) | 8M/9F) | |
| Moderate | Jumping (athletics) (1F) | | Handball | Skiing | Basketball | 165 |
| 20-50% | Rugby | Aquatics | (19M/3F) | Cross | (3M) | (70% M) |
| MVC) | (15M) | (3M/2F) | Hockey | Country | Swimming | |
| | Athletics other | Sprint | (4M/1F) | (3M/1F) | (25M/16F) | |
| | (41M/26F) | (2M) | | | | |

| Low | Baseball (2M) | Long-Distance running | | Tennis | 344 |
|---|---|---|---|---|---|
| (<20% | Volleyball (2M) | and marathon | | (3M/3F) | (95% M) |
| MVC) | | (37M/12F) | | | |
| | Table tennis (9M) | Soccer | Ultra- | Football | |
| | | (256M/1F) | running | (17M/1F) | |
| | | | (1F) | | |
| Total | 134 (73% M) | 662 (82% M) | | | 796 |
| | | | | | (81% M) |

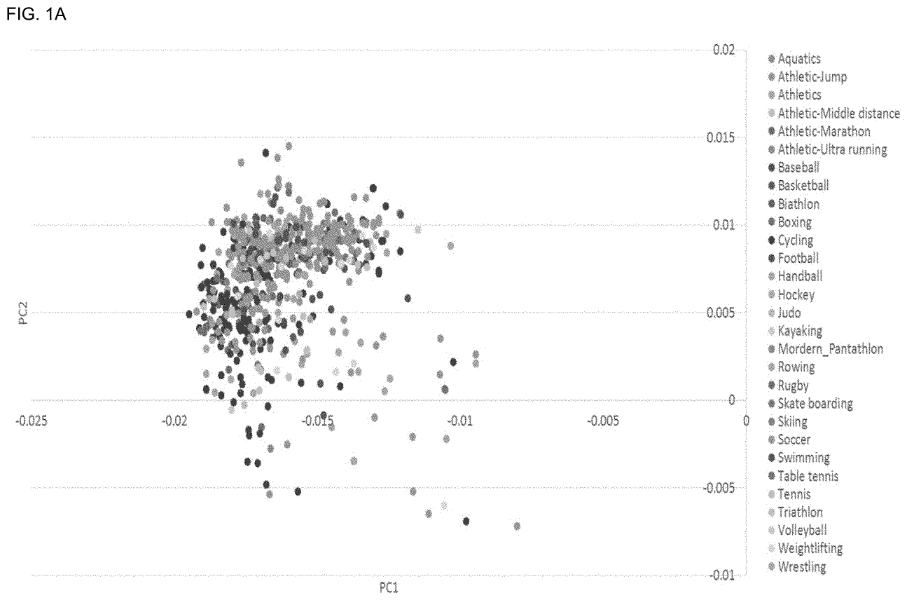

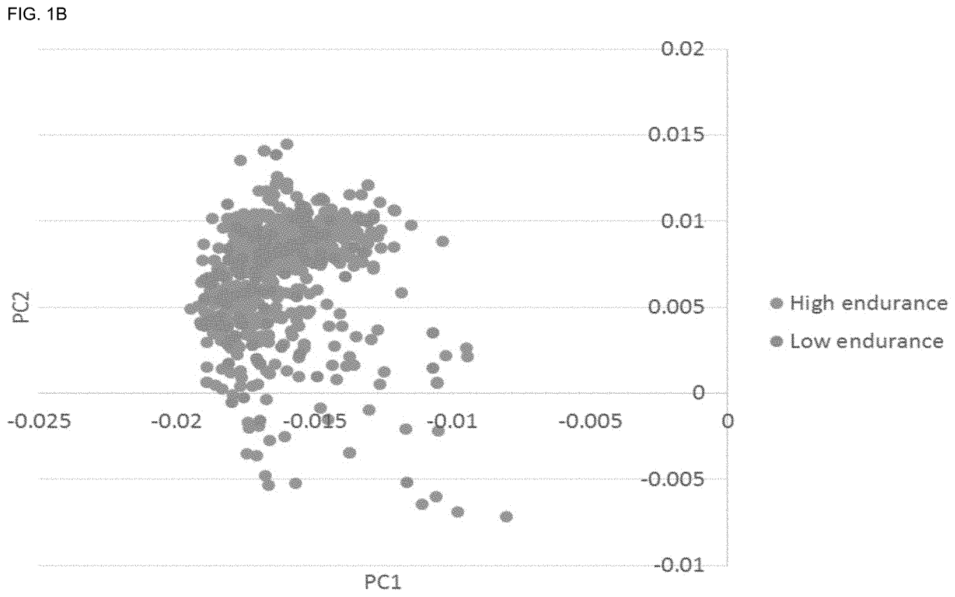

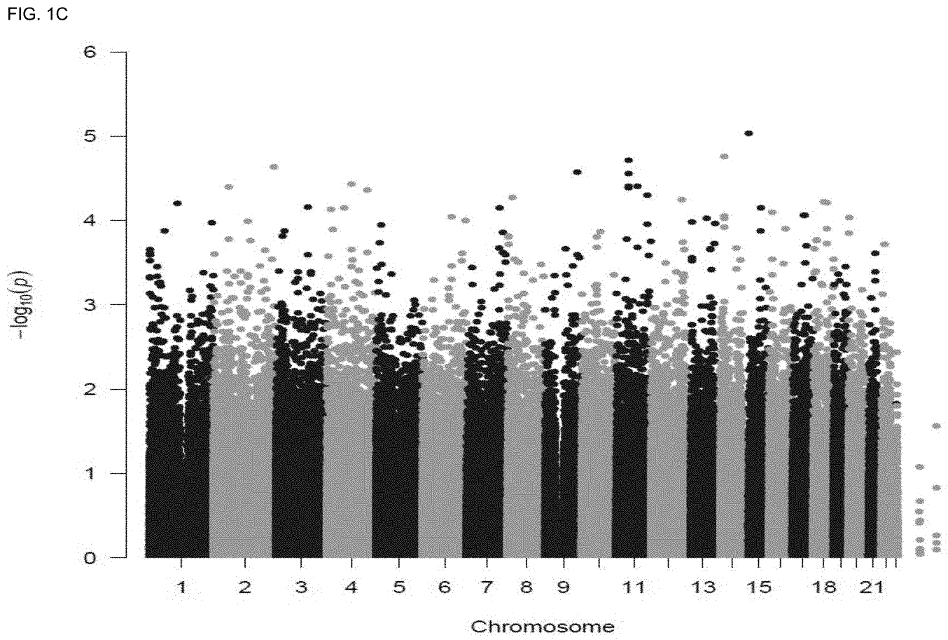

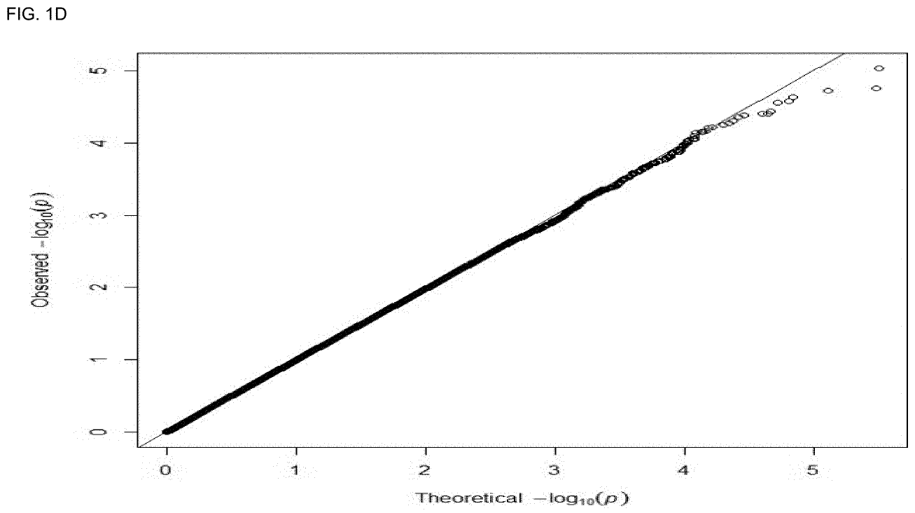

The principle component analysis (PCA) of the genotyping data revealed no influence of sport disciplines (FIG. 1A) or training modality (i.e. sports with low/moderate vs high aerobic component) (FIG. 1B) on genotype distribution. Following quality control data processing, genotyping of 341385 SNPs in 796 European elite athletes revealed several variants associated with endurance athlete status, but none reached GWAS level of significance. Table 2 shows top SNPs (P<10⁻⁴) with their odd ratios (OR) in relation to elite athletic endurance, location according to function genome variation server (GVS), gene name and minor allele frequency (MAF) in sports with high and low/moderate aerobic component. MAF in non-elite athletes from 1000 genome project were used as a reference. FIG. 1 shows Manhattan (C) and quartile-quartile (QQ) plots (D) of GWAS hits associated with endurance.

FIG. 1. GWAS data quality control. PCA shows no difference in the genotype distribution among sport disciplines (A) or between groups (sports with low/moderate vs high aerobic component) (B) Manhattan (arrow indicates significant SNPs identified in meta-analysis) (C) and Quantile-quantile (no evidence of genomic inflation, lambda GC=1.006) (D) plots illustrating GWAS results in association with endurance.

TABLE 2
Top GWAS SNPs associated with Endurance athlete status from
the discovery study.

| | | | | | | | | | | | | MAF- | |
|---|---|---|---|---|---|---|---|---|---|---|---|---|---|
| | | | | | | | | | | | | Mod- | |
| | | | | | | | | | | | MAF- | erate/ | |
| | | | Refer- | | | | Stand- | | | | High | Low | MAF- |
| | Chromo- | | ence | Allele | | | ard | P | Function | Gene | aerobic | aerobic | non- |
| rsID | some | Position | Base | 2 | N | OR | Error | value | GVS | List | N = 652 | N = 134 | athletes |
| rs8029108 | 15 | 22945314 | C | T | 795 | 0.5293 | 0.1435 | 9.23 × 10⁻ [symbol] | intron | CYFIP1 | 0.4448 | 0.403 | G = 0.36 |
| kgp5680198 | 14 | 34627202 | C | T | 792 | 0.5161 | 0.1545 | 1.75 × 10⁻ [symbol] | intergenic | LOC102724945 | 0.2135 | 0.3248 | C = 0.27 |
| [symbol] 0838680 | 11 | 47275064 | A | G | 794 | 0.5268 | 0.1526 | 1.92 × 10⁻ [symbol] | intron | NR1H3 | 0.233 | 0.3498 | A = 0.35 |
| kgp2861067 | 2 | 234653039 | T | C | 796 | 0.2227 | 0.3561 | 2.34 × 10⁻ [symbol] | intron | UGT1A10 | 0.01815 | 0.0597 | T = 0.013 |
| kgp11512684 | 9 | 123798492 | A | G | 793 | 0.202 | 0.3808 | 2.65 × 10⁻ [symbol] | intron | C5 | 0.01364 | 0.04887 | A = 0.016 |
| rs1052373 | 11 | 47354787 | A | G | 795 | 0.5393 | 0.1475 | 2.81 × 10⁻ [symbol] | missense | MYBPC3 | 0.2764 | 0.3955 | T = 0.39 |
| rs17029031 | 4 | 94380515 | G | A | 795 | 0.3064 | 0.2886 | 3.68 × 10⁻ [symbol] | intron | GRID2 | 0.0287 | 0.09398 | G = 0.09 |
| rs1949886 | 11 | 80311086 | A | G | 795 | 4.346 | 0.3573 | 3.92 × 10⁻ [symbol] | intergenic | none | 0.1329 | 0.03731 | A = 0.15 |
| rs7120118 | 11 | 47280290 | C | T | 798 | 0.5405 | 0.1475 | 3.97 × 10⁻ [symbol] | intron | NR1H3 | 0.2886 | 0.3881 | C = 0.38 |

[symbol]

indicates data missing or illegible when filed

Replication of Endurance SNPs in Russian and Japanese Elite Athlete Cohorts

Replication of results was performed by comparing the frequencies of the most significant SNPs (P<E-5) in 242 elite Russian high and 168 low/moderate aerobic athletes, and in 60 elite Japanese endurance athletes and 406 controls. Out of the 9 top SNPs identified from the GWAS discovery stage, the rs1052373 (MYBPC3) and rs7120118 (NR1H3) showed significant association with endurance in Russian and Japanese (p<0.05). However, the association was driven by a dominant model since results of this analysis showed over representation for rs1052373 GG and rs7120118 TT genotypes in the high endurance group. A subsequent meta-analysis has confirmed the over representation of the rs1052373 GG and rs7120118 TT genotypes in high endurance sports at genome-wide and Bonferroni levels of significance (1.43×10⁻⁸ and 1.66×10⁻⁷, respectively) (Table 3). The combined analysis showed no evidence of heterogeneity and direction of association was similar in all three cohorts. Table S1 shows the same associations using an additive model.

TABLE 3
SNPs associated with Endurance athlete status from the discovery,
replication and meta-analysis.

| | | | | GWAS | | Russian | | Japanese | | Combined | | | |
|---|---|---|---|---|---|---|---|---|---|---|---|---|---|
| | | | | | OR | | OR | | OR | | OR | | |
| | | | | | (95% | | (95% | | (95% | | (95% | | |
| Chr | SNP | RG | Position | P | Cl) | P | Cl) | P | Cl) | P | Cl) | I² | P [symbol] |
| 11 | rs1052373 | GG | 47,246,397- | 5.48 × 10⁻ [symbol] | 2.61 | 0.01 | 1.67 | 0.003 | 2.92 | 1.43 × 10⁻ [symbol] | 2.17 | 35 | 0.2 |
| | | | 47,360,412 | | (1.7- | | (1.1- | | (1.4- | | (1.7- | | |
| | | | | | 3.9) | | 2.5) | | 6.1) | | 2.8) | | |
| 11 | rs7120118 | TT | 47,246,397- | 1.26 × 10⁻ [symbol] | 2.49 | 0.02 | 1.64 | 0.035 | 2.48 | 1.66 × 10⁻⁷ | 2.07 | 12 | 0.3 |
| | | | 47,356,870 | | (1.7- | | (1.1- | | (1.1- | | (1.6- | | |
| | | | | | 3.8) | | 2.5) | | 5.6) | | 2.7) | | |

OR, odds ratio for the risk genotype; CI, confidence interval; I2, heterogeneity statistics; Phet, P value for heterogeneity [symbol]
indicates data missing or illegible when filed

TABLE S1
SNPs associated with Endurance athlete status from the
discovery, replication and meta-analysis (Additive model).

| | | | GWAS | | Russian | | Japanese | | Combined | | | |
|---|---|---|---|---|---|---|---|---|---|---|---|---|
| Chr | SNP | RA | P | OR | P | OR | P | OR | P | OR | I² | P [symbol] |
| 11 | rs1052373 | G | 2.81 × 10⁻ [symbol] | 1.85 | 0.02 | 1.41 | 0.02 | 1.18 | 7.95 × 10⁻⁶ | 1.62 | 44 | 0.2 |
| | | | | (1.39- | | (1.05- | | (0.79- | | (1.23- | | |
| | | | | 2.48) | | 1.89) | | 1.78) | | 1.77) | | |
| 11 | rs7120118 | T | 3.98 × 10⁻ [symbol] | 1.83 | 0.41 | 1.40 | 0.29 | 1.25 | 6.35 × 10⁻ [symbol] | 1.53 | 26 | 0.3 |
| | | | | (1.37- | | (1.05- | | (0.82- | | (1.25- | | |
| | | | | 2.45) | | 1.88) | | 1.91) | | 1.79) | | |

OR, odds ratio for the risk genotype; CI, confidence interval; I2, heterogenerity statistics; Phet, P value for heterogenerity. [symbol]
indicates data missing or illegible when filed

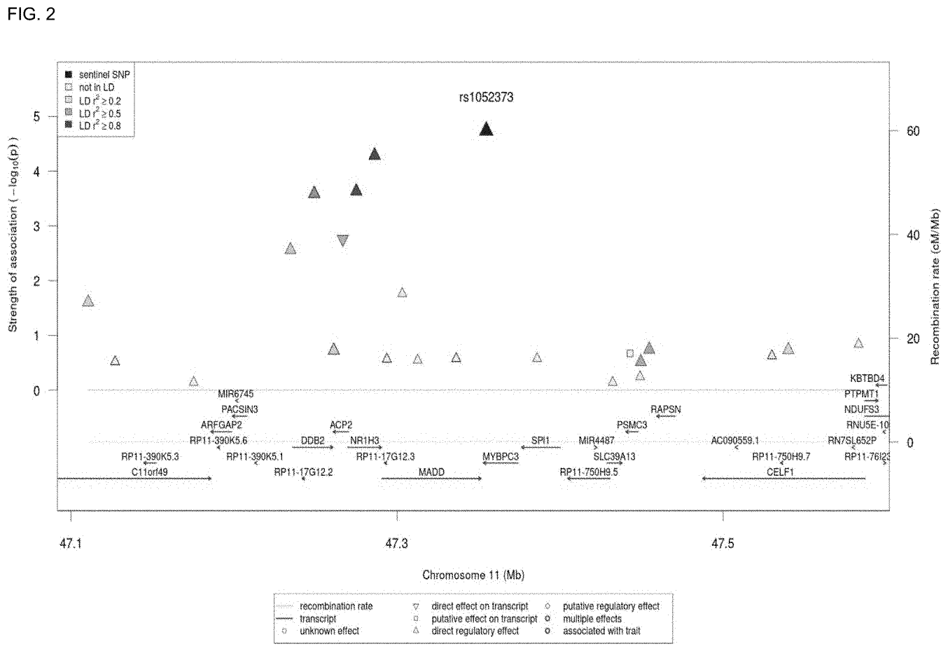

FIG. 2 shows a regional association plot for the region around rs1052373. The colors correspond to different LD thresholds, where LD is computed between the sentinel SNP (lowest p-value, colored in blue) and all SNPs. Shapes of markers correspond to their functionality as described in the legend.

To validate the potential functionality of the identified GWAS SNPs, association of the identified two SNPs (rs1052373 G and rs7120118 T alleles) with VO₂ₘₐₓ was investigated in a subgroup of the Russian replication cohort in which VO₂ₘₐₓ data was available. This included 32 elite Russian long-distance athletes (19 biathletes, 13 cross-country skiers; 17 females, age 23.5 (3.5) years; 15 males, age 21.3 (4.1) years). The rs1052373 GG carriers had significantly greater VO₂ₘₐₓ than carriers of the AA+AG (P=0.005 adjusted for sex). Similarly, rs7120118 TT carriers showed a trend of higher VO₂ₘₐₓ than carriers of the CC+CT (P=0.053 adjusted for sex).

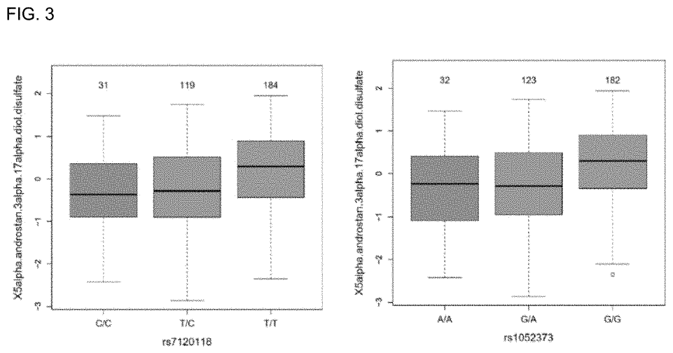

For further validation of the potential functionality of the identified GWAS SNPs, metabolomics of 750 metabolites was carried out in a subset of the discovery cohort (n=490) and enriched metabolic pathways associated with the rs1052373 G allele and rs7120118 T alleles were determined (Table 4). Among the metabolic pathways associated with rs56330321 and rs7120118, various lipids and amino acids were significantly altered by their genotypes. However, only 5alpha-androstan-3alpha, 17alpha-diol disulfate reached Bonferroni level of significance (Table 4), exhibiting higher levels in rs1052373 GG and rs7120118 TT carriers compared to AA+AG and CC+TC carriers, respectively (FIG. 3). FIG. 3 shows boxplots representing levels of 5alpha-androstan-3alpha, 17alpha-diol disulfate in rs7120118 and rs1052373 genotype groups.

TABLE 3
Metabolites that belong to the significantly enriched phospholipids
pathway Top metabolites associated with significant SNPs.

| SNP | Beta | SE.Beta | P | Metabolites | SUPER_PATHWAY | SUB_PATHWAY |
|---|---|---|---|---|---|---|
| | −0.36 | 0.03 | 1.82 × 10⁻⁵ | 5alpha-androstan- | Lipid | Androgenic Steroids |
| | | | | 3alpha, 17alpha-diol disulfate | | |
| | −0.25 | 0.07 | 0.000248 | 2-hydroxy-3-methylvalerate | Amino Acid | Leucine, Isoleucine and Valine |
| | | | | | | Metabolism |
| | −0.23 | 0.07 | 0.000879 | alpha-hydroxylsovalerate | Amino Acid | Leucine, Isoleucine and Valine |
| | | | | | | Metabolism |
| | 0.31 | 0.09 | 0.000928 | xylose | Carbohydrate | Pentose Metabolism |
| rs1052373 | −0.23 | 0.07 | 0.001226 | N1-methylinosine | Nucleotide | Purine Metabolism. |
| | | | | | | (Hypo)Xanthne/Inosine containing |
| | −0.23 | 0.07 | 0.001315 | palmitoleoylcarnitine (C16:1)* | Lipid | Fatty Acid Metabolism(Acyl Carnitine) |
| | −0.23 | 0.07 | 0.001509 | 2-hydroxyadipate | Lipid | Fatty Acid, Dicarboxylate |
| | −0.22 | 0.07 | 0.001516 | 2-methylcitrate/homocitrate | Energy | TCA Cycle |
| | −0.21 | 0.07 | 0.001933 | myristoleoylcarnitine (C14:1)* | Lipid | Fatty Acid Metabolism(Acyl Carnitine) |
| | −0.33 | 0.08 | 5.17 × 10⁻ [symbol] | 5alpha-androstan- | Lipid | Androgenic Steroids |
| | | | | 3alpha, 17alpha-diol disulfate | | |
| | −0.27 | 0.07 | 0.000136 | 2-hydroxy-3-methylvalerate | Amino Acid | Leucine, Isoleucine and Valine |
| | | | | | | Metabolism |
| | −0.24 | 0.07 | 0.000582 | alpha-hydroxylsovalerate | Amino Acid | Leucine, Isoleucine and Valine |
| | | | | | | Metabolism |
| | −0.24 | 0.07 | 0.000715 | N1-methylinosine | Nucleotide | Purine Metabolism. |
| | | | | | | (Hypo)Xanthne/Inosine containing |
| rs7120118 | 0.31 | 0.09 | 0.001004 | xylose | Carbohydrate | Pentose Metabolism |
| | −0.23 | 0.07 | 0.001527 | 2-hydroxyadipate | Lipid | Fatty Acid, Dicarboxylate |
| | 0.28 | 0.09 | 0.001966 | 5-acetylamino-6-formylamino-3- | Xenobiotics | Xanthine Metabolism |
| | | | | methyluracil | | |
| | −0.22 | 0.07 | 0.002116 | alpha-hydroxylsocaproate | Amino Acid | Leucine, Isoleucine and Valine |
| | | | | | | Metabolism |
| | −0.22 | 0.07 | 0.002216 | 2-methylcitrate/homocitrate | Energy | TCA Cycle |
| | −0.22 | 0.07 | 0.002266 | glycerol | Lipid | Glycerolipid Metabolism |

[symbol]

indicates data missing or illegible when filed

### DISCUSSION

Genetic predisposition into cardiorespiratory fitness and response to exercise training has been previously described. Since endurance performance sports are characterized by increased cardiorespiratory capacity, genetic predisposition into elite endurance performance is also expected to be genetically influenced. However, genetic studies of elite athletic endurance showed inconsistent results. The aims of this study were to carry out the largest GWAS study of elite European athletes to date using a unique SNP microarray that is enriched with genes involved in different metabolic pathways with direct influence on various physiological pathways characteristic of elite athletes. GWAS results have revealed a number of novel SNPs associated with endurance but none reached the GWAS level of significance. Replication of the top identified SNP associations in two independent cohorts of elite athletes from Russia and Japan has confirmed the association of rs7120118 and rs1052373 with endurance athlete status. Subsequent meta-analysis of the three cohorts has revealed for the first time that both SNPs were associated with endurance athlete status at genome-wide and Bonferroni level of significance, respectively. Functional validation has revealed the association of the two SNPs with increased Vo2max and levels of the testosterone precursor 5alpha-androstan-3alpha, 17alpha-diol disulfate.

The top identified GWAS significant SNP (rs1052373) is located within MYBPC3 gene. MYBPC3 codes for a myosin-associated protein expressed in the cross-bridge-bearing zone (C region) of A bands in striated muscle. The phosphorylation of MYBPC3 protein modulates cardiac contraction. Mutations in MYBPC3 were previously associated with a lower super-relaxed state in patients with hypertrophic cardiomyopathy (HCM). Intense exercise can trigger heart remodeling to compensate for the elevations in blood pressure or volume by increasing muscle mass. Hence, hearts of the endurance athletes typically exhibit an eccentric cardiac hypertrophy with increased cavity dimension and wall thickness, which is influenced by the type of sport performed. As a result, the endurance-trained heart can deliver a large maximal systolic volume (35% larger than untrained heart) in order to produce a large cardiac output. Since carriers of the GG allele exhibit a benign phenotype of HCM according to NIH's ClinVar database, the mild phenotype may be enhancing exercise-triggered physiological adaptations. The seemingly dominant effect of rs1052373 GG on increased Vo₂ₘₐₓ and endurance may support this added advantage although more studies are needed to confirm this finding. These adaptations, however, might be associated with a greater risk of cardiovascular disease. Indeed, we have recently shown that endurance athletes with high cardiovascular demand (higher blood pressure and stroke volume) show metabolic signature consistent with higher risk of cardiovascular disease. When investigating the expression quantitative trait loci (eQTLs) associated with rs1052373 in the peripheral blood monocytes, a number of genes was identified including SPI1, MYBPC3, MADD, ACP2 and NR1H3. Interestingly, eQTL (GTEx) showed that rs1052373 polymorphism is associated with expression level of MADD and ACP2 in heart, but not MYBPC3. Since MAP kinase plays an important role of cardiac hypertrophy, the association between rs1052373 polymorphism and Vo2max and endurance may also be explained by MADD expression, although this needs further validation. Information related to function and associated diseases with these genes are summarized in Table S2.

TABLE S2
List of genes in eQTL with rs1052373 in the peripheral blood
monocytes including their function and associated diseases.

| | Minor | | | | |
|---|---|---|---|---|---|
| SNP | Allele | Gene name | P-value | Gene Function | Associated diseases |
| [symbol] | T | Spi-1 (Spi-1 | 3.3 [symbol] × | An ETS-domain transcription factor that | Inflammatory Dia [symbol] and |
| TT | | Proto- [symbol] | 10 [symbol] | activates gene expression during myeloid | Primary M [symbol] B-Cell |
| | | | | and B-lymphoid cell development | Lymphoma |
| | | Myosin Bind- | 1.200 [symbol] × | A myosin-associated protein found in the | Cardiomyopathy, Familial |
| | | ing Protein C. | 10 [symbol] | cross-bridge-bea [symbol] ing zone (C region) | Hypertrophic [symbol] and Left |
| | | Cardiac | | of A [symbol] in [symbol] muscle. [symbol] phos [symbol] | Ven [symbol] cular Noncom- |
| | | (MYBPC3) | | modulates cardiac contraction | paction |
| | | MAP Kinese | | A death [symbol] -containing adaptor protein | Diastolic Heart Failure & |
| | | Activating | | that interacts with the death domain of | cardiac hypertrophy |
| | | Death Domain | | TNF-alpha receptor 1 to activate [symbol] gen- | |
| | | (MADD) | | activated protein kinase (MAPK) and | |
| | | | | propagate the apoptotic signal. | |
| | | ACP2 (Acid | 2.1617 × | A histidine acid phosphatase that | Bone structure alterations, |
| | | Phosphatase 2. | 10 [symbol] | hydro [symbol] orthophosphoric monoesters to | lysosomal storage defects, |
| | | Lyso [symbol] l) | | alcohol and phosphate. | and an increased tendency |
| | | | | | towards seizures |
| | | N [symbol] H3 | 4. [symbol] × | A nuclear receptor that works as a key | Multiple [symbol] erosis and |
| | | (Nuc [symbol] | 10 [symbol] | regulator of macrophage function, | [symbol] rotendinous |
| | | Receptor | | controlling transcriptional programs | Xanthomatosis. Among its |
| | | Subfamily | | involved in lipid homeostasis and | related pathways are Lipo- |
| | | 1 Group H | | inflammation. Plays an important role in | protein metabolism and |
| | | Member [symbol] ) | | the regulation of cholesterol homeostasis. | Nuclear Receptors in Lipid |
| | | | | Liver X receptors regulate adrenal | Metabolism and Toxicity |
| | | | | steroidogenesis. | |

[symbol]

indicates data missing or illegible when filed

The other significant association was between rs7120118 TT carriers and high endurance. Rs7120118 is located in NR1H3 gene that codes for a nuclear receptor regulating macrophage function, lipid homeostasis and inflammation. NR1H3, also known as liver X Receptor Alpha (LXRA), plays an important role in the regulation of cholesterol homeostasis including adrenal steroidogenesis. The association of rs7120118 with high endurance could be reflecting the high linkage disequilibrium (r²=0.89, p<0.0001) between rs7120118 TT and the potentially functional rs1052373 GG. It could, however, be related to increased synthesis of the testosterone precursor 5alpha-androstan-3alpha, 17alpha-diol disulfate since NR1H3 regulates hypothalamo-pituitary-adrenal steroidogenesis. Indeed, we have previously shown that high-endurance athletes exhibit elevated levels of several sex hormone steroids involved in testosterone synthesis including 5alpha-androstan-3alpha, 17alpha-diol disulfate with implication on improving performance due to enhanced glucose metabolism and protein synthesis in the muscle. The functional relevance of these associations remains to be further validated.

Study limitations: The lack of information about participants and the heterogeneity of their sport groups were major limitations of this study. Additionally, the association of rs1052373 and rs7120118 SNPs with endurance only reached GWAS and Bonferroni level of significance, respectively, after conducting meta-analysis. Neither of the two SNPs reached GWAS or Bonferroni level of significance in the discovery of replication cohorts independently, therefore the association was not replicated. This maybe related to the underpowered nature of the study to detect variants with small effect size. To overcome these limitations and to increase the power of the study, genotyping was compared between athletes who belong to high endurance versus moderate endurance performance sports instead of power versus endurance due to the overlap between the two classes as per Mitchell's categorization. Other limitations included using add-on replication studies (Russian and Japanese cohorts) rather than using a carefully designed replication, despite the differences in the analyzed phenotype (high vs low VO₂ₘₐₓ in European and Russian participants whereas endurance vs controls in Japanese participants) among the studies. However, differences were confirmed in each study separately and the subsequent meta-analysis confirmed the significance of the association of the two SNPs with endurance. Another limitation is related to attributing the association of rs1052373 with endurance to MYBPC3 function, although the SNP is in eQTL with other potentially relevant genes that contain other SNPs in high linkage disequilibrium with rs1052373. However, as rs1052373 is located within and is in eQTL with MYOBPC3, we believe the association could potentially be driven through MYBPC3 function, although validation in other studies is warranted to confirm functional association. Finally, when the additive model used in the discovery GWAS was adopted in replication studies (Russian and Japanese cohorts), the association did not reached GWAS level of significance in the meta analysis, despite reaching Bonferroni significance (Table S1). Whereas the dominant model reached GWAS significance in the meta analysis and it corresponded well with the autosomal dominant mode of inheritance of MYBPC3 in hypertrophic cardiomyopathy, therefore it was adopted in the replication studies.

### CONCLUSIONS

This study reports the first GWAS significant SNP (rs1052373) in MYBPC3 in association with endurance athlete status with a direct relevance to cardiac hypertrophy and contraction. The SNP is associated with increased VO2max and elevated levels of the testosterone precursor androstenediol (3beta, 17beta) disulfate, both phenotypes that potentially contribute to the superior performance of endurance athletes. This study also identifies a second SNP (rs7120118) associated with endurance at Bonferroni level of significance in NR1H3. This SNP could be either working independently of rs1052373 through influencing steroidogenesis or could be acting as a marker of rs1052373. Further investigations of the functional relevance of the identified SNPs and associated metabolites in relation to enhanced athletic performance are warranted.

Methods

The aim of this study was to investigate the genetic predisposition to elite athletic endurance through conducting the largest GWAS in elite athletes to date, followed by functional validation through aerobic capacity testing and metabolomics analysis to shed light on the underlying mechanisms of genetic associations.

Participants

Discovery Study

Seven hundred and ninety six consented European international-level athletes (645 males, 151 females) from different sports disciplines who participated in national or international sports events and tested negative for doping substances at anti-doping laboratories in Qatar (ADLQ) and Italy (FMSI) were included in this study. No other information of participants was available due to the strict anonymization process undertaken by the anti-doping laboratories. This study was performed in line with the World Medical Association Declaration of Helsinki—Ethical Principles for Medical Research Involving Human Subjects. All protocols were approved by the Institutional Research Board of ADLQ (F2014000009). Athletes were dichotomized into groups with different aerobic (dynamic) and power (static) components (Table 1) based on their sport types. Table 1 further lists the number of participants based on various analyses as per sport type in each class/group and their genders.

Replication Studies

The first replication study involved 410 Russian athletes (187 females, age 25.3 (4.1) years, 223 males, age 25.7 (4.3) years). Athletes were dichotomized into two groups with different aerobic (dynamic) and power (static) components based on their sport types.

Group 1 (242 athletes with high aerobic component) included biathletes (n=19), cross-country skiers (n=16), 800-10000 m runners (n=9), rowers (n=9), kayakers (n=30), canoers (n=8), speed skaters (n=12), short-trackers (n=3), swimmers (n=38), cyclists (n=5), race walkers (n=6), boxers (n=43), badminton players (n=11), basketball players (n=6), water polo players (n=12), football players (n=9), and ice hockey players (n=6).

Group 2 (168 athletes with low aerobic component) included 100-400 m runners (n=8), wrestlers (n=44), alpine skiers (n=2), sailors (n=2), synchronized swimmer (n=1), taekwondo athletes (n=5), baseball players (n=10), volleyball players (n=19), table tennis players (n=5), softball players (n=5), rhythmic gymnasts (n=7), chess players (n=5), throwers (n=6), athletics jumpers (n=16), ski jumpers (n=2), weightlifters (n=25), figure skaters (n=6).

All athletes were Olympic team members (International level; all Caucasians of Eastern European descent) who have tested negative for doping substances. The Russian study was approved by the Ethics Committee of the Federal Research and Clinical Center of Physical-chemical Medicine of the Federal Medical and Biological Agency of Russia. Written informed consent was obtained from each participant. The study complied with the guidelines set out in the Declaration of Helsinki and ethical standards in sport and exercise science research. The experimental procedures were conducted in accordance with the set of guiding principles for reporting the results of genetic association studies defined by the STrengthening the REporting of Genetic Association studies (STREGA) Statement.

The second replication study involved endurance athletes (n=60) and controls (n=406) from Japan. All endurance athletes were track and field competitors who participated in endurance events from 800 m to marathon. In addition, all athletes were international athletes who had competed at major international competitions. All controls were healthy Japanese individuals. All subjects gave written informed consent before their inclusion in the study. The study protocols were approved by the ethics committee of the Juntendo University and was conducted according to the Declaration of Helsinki.

Aerobic Capacity Testing

VO₂ₘₐₓ in biathletes and cross-country skiers was determined using an incremental test to exhaustion on a treadmill HP Cosmos (Germany). The initial speed was 7 km/h, the increment was 0.1 km/h every 10 seconds. VO₂ₘₐₓ was determined breath by breath using a MetaMax 3B-R2 gas analysis system. VO₂ₘₐₓ was recorded as the highest mean value observed over a 30 s period.

Genotyping

Discovery Study

DNA was extracted from leukocytes (venous blood) samples from all participants using DNeasy Blood & Tissue kit (Qiagen) following manufacturer's instructions. The concentration and the quality of DNA were assessed using the Nanodrop (Thermo Fisher) and Qubit Fluorometer (Invitrogen) to ensure sufficient amount and quality of DNA were obtained for genotyping. Illumina Drug Core array-24 BeadChips was chosen for the genotyping of 476,728 SNPs in the 837 European elite athletes collected for Anti-Doping analysis (discovery cohort). This array contains over 240,000 highly-informative genome-wide tag SNPs and a novel ˜200,000 custom marker set designed to support studies of drug target validation and treatment response. The assay required 200 ng of DNA sample as input with a concentration of at least 50 ng/μl. All further procedures were performed according to the instructions of Infinium HD Assay according to manufacturer's instructions. Briefly, 4 μl of obtained DNA was mixed with Illumina amplification reagents and incubated overnight at 37° C. in hybridization oven. On the second day, enzymatic reagents were used to fragment the amplified DNA then precipitated by centrifugation. Subsequently, re-suspended pellet was loaded in the beadchip then incubated overnight at 48° C. in hybridization oven. On the third day, beadchips underwent enzymatic base extension and fluorescent staining. Lastly, after coating, the beadchips were imaged using iScan.

Replication Studies

Molecular genetic analysis in Russian cohorts was performed with DNA samples obtained from leukocytes (venous blood). Four mL of venous blood were collected in tubes containing EDTA (Vacuette EDTA tubes, Greiner Bio-One, Austria). Blood samples were transported to the laboratory at 4° C. and DNA was extracted on the same day. DNA extraction and purification were performed using a commercial kit according to the manufacturer's instructions (Technoclon, Russia) and included chemical lysis, selective DNA binding on silica spin columns and ethanol washing. Extracted DNA quality was assessed by agarose gel electrophoresis at this step. HumanOmni1-Quad BeadChips (Illumina Inc, USA) were used for genotyping of 1,140,419 SNPs in athletes and controls. The assay required 200 ng of DNA sample as input with a concentration of at least 50 ng/μl. Exact concentrations of DNA in each sample were measured using a Qubit Fluorometer (Invitrogen, USA). All further procedures were performed according to the instructions of Infinium HD Assay. For the second replication study, total DNA was isolated from saliva or venous blood using Oragene⋅DNA Collection Kits (DNA genotek, Ontario, Canada) or QIAamp DNA blood Maxi Kit (QIAGEN, Hilden, Germany), respectively. The total DNA content was measured using a NanoDrop 8000 spectrophotometer (Thermo Fisher Scientific, MA, USA). Subsequently, DNA samples were adjusted to a concentration of 50 ng/μL with TE buffer and were stored at 4° C. Total DNA samples were genotyped for more than 700,000 markers using the Illumina® HumanOmniExpress Beadchip.

Data Extraction and SNP Identification

Raw data was extracted, peak-identified and QC processed using Illumina iScan hardware and software. These systems are built on a web-service platform utilizing Microsoft's NET technologies, which run on high-performance application servers and fiber-channel storage arrays in clusters to provide active failover and load-balancing.

Metabolomics

Screening of serum metabolites was performed in 490 elite athletes (Table S3) using protocols established at Metabolon, Durham, N.C., USA. The platform utilizes Waters ACQUITY ultra-performance liquid chromatography (UPLC) and a Thermo Scientific Q-Exactive high resolution/accurate mass spectrometer interfaced with a heated electrospray ionization (HESI-II) source and Orbitrap mass analyzer operated at 35,000 mass resolution. Detailed protocol and QC measures were previously published.

Table S3. Classification of GWAS participants according to sports classes. Distribution of elite athletes in various categories based on sport type-associated peak dynamic (maximal oxygen uptake percentage; VO₂ₘₐₓ) and peak static (maximal voluntary muscle contraction percentage; MVC) components achieved during competition as described previously.

TABLE S3

| | | Endurance | |
|---|---|---|---|
| (4.b) | | Moderate | High |
| Replication | | (40-70% VO2max) | (>70% VO2max) |
| Power | High | Wrestling (3 M), Judo | Boxing (1 M/16 F), |
| | (>50% | (3 M) | Heptathlon (1 M), Rowing |
| | MVC) | | (6 M/7 F), Cycling |
| | | | (31 M/4 F) |
| | Moderate | Athletics (15 M/22 F), | Athletics 200-800 m |
| | (20-50% | Rugby (16 M), Triple | (4 M), Hockey (1 F), |
| | MVC) | Jump (1 M) | Skiing Cross Country |
| | | | (1 M), Basketball (3 M), |
| | | | Swimming (22 M/16 F) |
| | Low | Baseball (2 M), | Tennis (1 M/1 F), Soccer |
| | (<20% | Volleyball (1 M) | (315 M), Athletics 1500- |
| | MVC) | | 3000 m (3 M) |

Statistical Analysis

Following genotyping using Illumina's Drug Core SNP array, analysis was performed using Plink v1.9. Quality control measures were applied to the genotype data set (837 samples with 476,728 SNPs) to exclude samples with low genotype call rate (<95%; n=21) or excess heterozygosity (n=3). Accordingly, SNPs with a genotype call rate <98% (n=35,736), minor allele frequency <1% (n=88,033), or deviating from Hardy-Weinberg equilibrium (P<10⁻⁶; n=11,574)) were excluded. After filtering the data with the above criteria, 341,385 SNPs were used in analysis. Population background was determined using principal component analysis (PCA) in comparison to samples from HapMap project and only samples with European ancestry were included in the analysis. Population ancestry outliers (n=17) were removed based on deviation from the mean (±4SD) of the first two population principal components. The final file used for analysis contained 796 samples. The analysis in European and Russian cohorts was performed using linear or logistic regression models. A model incorporating sports grouped by training modalities (i.e. sports with high vs. low/moderate aerobic component) was used for the discovery cohort after incorporating gender and PCA components 1, 2, 3 & 4 as covariates in the model. A stringent Bonferroni level of significance of p<=0.05/341385=1.46×10⁻⁷ was used to define significant associations. The analysis was repeated after adjusting for MVC and resulted in essentially similar results both for allelic and dominant model (data not shown). Out of the 9 top SNPs that were selected in the discovery cohort (P<1.0E-04), only 6 were validated in Russian cohort (3 were not found in the Russian of Japanese chips), but only two replicated. These two were then also replicated in Japanese cohort and meta-analysis was performed for only these two SNPs. To perform the meta-analysis, the Cochrane Review Manager version 5.3 was used. Random and fixed effect models were applied. The heterogeneity degree between the studies was assessed with the I² statistics. Associations between SNPs and metabolite levels were computed using Im function in R (version 3.3.1) while correcting for gender, hemolysis and PCA. An additive inheritance model was used (SNPs were coded as 0, 1, 2 according to their genotype group. Pathway enrichment analyses were carried out using Chi square tests to identify pathways with enriched metabolites ranked by p-value from the linear model since Bonferroni level of significance was not observed. Genetic loci were investigated for known eQTLs, mQTLs and functional associations using several databases including: SNIPA http://snipa.helmholtzmuenchen.de/snipa/, henoScanner V2 A database of human genotype-phenotype associations http://www.phenoscanner.medschl.cam.ac.uk/, GTEx portal (version 2.1, Build #201) www.gtexportal.org, OMIM www.omim.org, Overview of Bravo variant server resources https://bravo.sph.umich.edu/freeze3a/hg19/, GnomAD http://qnomad.broadinstitute.org/.

### Example 2

From our data (Front. Genet. 11:595. doi: 10.3389/fgene.2020.00595), we have identified 6 SNPs that showed significant association with endurance in a case-control design (endurance athletes vs controls/sprinters) in European and Russian elite athletes (in collaboration with Dr Ildus Ahmetov, Liverpool John Moores University).
1. rs1052373 G
2. rs6455978 T
3. rs10036834 A
4. rs2292434 G
5. rs2477838 G
6. rs4824047 A

These SNPs were found to be:
1. Over-represented in European athletes with high aerobic component;
2. Over-represented in Russian endurance athletes compared to controls/sprinters;
3. Associated with increased VO2max in Russian athletes.

When compared to UK Biobank cohort, we identified the following:
1. rs6455978 Decreased Frequency of tiredness/lethargy in last 2 weeks P=0.032;
2. rs10036834 Increased Mean corpuscular hemoglobin concentration P=0.026; increased trunk fat-free mass p=0.043;
3. rs2292434 Low heart rate P=0.02; higher frequency of other exercises in last 4 weeks P=0.047; increased forced vital capacity P=0.038;
4. rs2477838 Decreased Frequency of tiredness/lethargy in last 2 weeks P=0.0071;
5. rs4824047 Rare occurrence of chronic fatigue syndrome P=0.011.

Thus, UK Biobank data supports our hypothesis that these SNPs may be associated with endurance.

We have then calculated the polygenic score analysis using genotyping data of these 6 SNPs (weighted by the effect size from the predictive model) in 693 elite athletes and used 75th percentile of the polygenic score (0.56) as a cutoff point to call an elite athlete high or low endurance:

Statistics
Polygenic Score

| N | Valid | 693 |
|---|---|---|
| | Missing | 0 |
| Mean | | −.01330 |
| Median | | .00000 |
| Std. Deviation | | .791814 |
| Skewness | | .039 |
| Std. Error of Skewness | | .093 |
| Percentiles | 25 | −.50170 |
| | 50 | .00000 |
| | 75 | .56361 |
| | 80 | .64336 |
| | 90 | .94689 |

Accordingly, we tested the sensitivity by ROC analysis using two independent elite athletes' cohorts: European athletes (n=666), non-European athletes (n=130) then the two combined cohorts as follows:

| | | |
|---|---|---|
| Predicted low | Predicted high | |
| endurance | endurance | Total |

Europeans elite athletes

| | | | |
|---|---|---|---|
| Observed low | 92 | 11 | 103 |
| endurance | | | |
| Observed high | 420 | 143 | 563 |
| endurance | | | |
| Total | 512 | 154 | 666 |

| | |
|---|---|
| Sensitivity | 0.93 |

Non-Europeans elite athletes

| | | | |
|---|---|---|---|
| Observed low | 26 | 4 | 30 |
| endurance | | | |
| Observed high | 71 | 29 | 100 |
| endurance | | | |
| Total | 79 | 33 | 130 |

| | |
|---|---|
| Sensitivity | 0.88 |

Combined European and non-European elite athletes

| | | | |
|---|---|---|---|
| Observed low | 118 | 15 | 133 |
| endurance | | | |
| Observed high | 491 | 172 | 663 |
| endurance | | | |
| Total | 609 | 187 | 796 |

| | |
|---|---|
| Sensitivity | 0.92 |

Based on the polygenic score of these 6 SNPs and the threshold based on the 75th percentile (0.56), we can now identify elite endurance athletes who carry these SNPs with 90% sensitivity.

We then designed a SNP chip containing our selected 6 SNPs in addition to previously reported 30 SNPs that were previously shown to be associated with endurance to be used for genotyping of endurance.

| | | | | | Endurance- |
|---|---|---|---|---|---|
| | Gene | Full name | Locus | Polymorphism | related allele |
| 1 | ACTN3 | Actinin alpha 3 | 11q13.1 | rs1815739 C/T | T |
| 2 | ACE | Angiotensin converting enzyme | 17q23.3 | rs4341 G/A | G |
| 3 | ADRB2 | Adrenoceptor beta 2 | 5q31-q32 | rs1042713 G/A | A |
| 4 | AGTR2 | Angiotensin [symbol] receptor type 2 | Xq22-q23 | rs11091046 A/C | C |
| 5 | AQP1 | Aquaporin 1 | 7p14 | rs1049305 C/G | C |
| 6 | AMPD1 | Adenosine monophosphate deaminase 1 | 1p13 | rs17602729 C/T | C |
| 7 | CKM | Creatine kinase M-type | 19q13.32 | rs8111989 A/G | A |
| 8 | COL5A1 | Collagen type V alpha 1 chain | 9q34.2-q34.3 | rs12722 C/T | T |
| 9 | FTC | FTC Alpha-Ketoglutarate Dependent | 16q12.2 | rs9939609 T/A | T |
| | | Dioxygenase | | | |
| 10 | GABPB1 | GA binding protein transcription factor | 15q21.2 | rs12594956 A/C | A |
| 11 | | subunit beta 1 | | rs7181866 A/G | G |
| 12 | GALNTL6 | Polypeptide N-acetylgalactosaminyl- | 4q34.1 | rs558129 T/C | C |
| | | transferase 6 | | | |
| 13 | GSTP1 | Glutathione S-transferase Pi 1 | 11q13.2 | rs1695 A/G | G |
| 14 | HFE | Homeostatic iron regulator | 6p21.3 | rs1799945 C/G | G |
| 15 | HIF1A | Hypoxia inducible factor 1 subunit alpha | 14q23.2 | rs11549465 C/T | C |
| 16 | LINC01060 | Long intergenic non-protein coding | 4q35.2 | rs2292434 A/C/G | G |
| | | RNA 1060 | | | |
| 17 | LINC01276 | Long intergenic non-protein coding | 6p13 | rs2477838 A/G | G |
| | | RNA 1276 | | | |
| 18 | MCT1 | Monocarboxylate transporter 1 | 1p12 | rs1049434 A/T | T |
| 19 | MYBPC3 | Myosin Binding Protein C3 | 11p11.2 | rs1052373 A/G | G |
| 20 | NFATC4 | Nuclear factor of activated T cells 4 | 14q11.2 | rs2229309 G/C | G |
| 21 | NFIA-AS2 | NFRA antisense RNA 2 | 1p31.3 | rs1572312 C/A | C |
| 22 | NOS3 | Nitric oxide synthase 3 | 7q36 | rs2070744 T/C | T |
| 23 | PPARA | Peroxisome proliferator activated | 22q13.31 | rs4253778 G/C | G |
| | | receptor alpha | | | |
| 24 | PPARGC1A | Peroxisome proliferative activated | 4p15.1 | rs8192678 G/A | G |
| | | receptor, gamma, coactivator 1 alpha | | | |
| 25 | PPARGC1B | Peroxisome proliferative activated | 5q32 | rs7732671 G/C | C |
| | | receptor, gamma, coactivator 1 beta | | | |
| 26 | RBFOX1 | RNA binding fox-1 homolog 1 | 16p13.3 | rs7191721 G/A | G |
| 27 | RNF130 | Ring finger protein 130 | 5q35.3 | rs10036834 G/A | A |
| 28 | SPEC | Striated Muscle Enriched Protein Kinase | 2q35 | rs7564858 G/A | G |
| 29 | TFAM | Transcription factor A, mitochondrial | 10q21 | rs1937 G/C | C |
| 30 | TSHR | Thyroid stimulating hormone receptor | 14q31 | rs7144481 T/C | C |
| 31 | UCP2 | Uncoupling protein 2 | 11q13 | rs660339 C/T | T |
| 32 | UCP3 | Uncoupling Protein 3 | 11q13 | rs1800849 C/T | T |
| 33 | VEGFA | Vascular endothelial growth factor A | 6p12 | rs2010963 G/C | C |
| 34 | VEGFR2 | Vascular endothelial growth factor | 4q11-q12 | rs1870377 T/A | A |
| | | receptor 2 | | | |
| 35 | None | None | 22p13 | rs4824047 G/A | A |
| 36 | None | None | 6p13 | rs6455978 T/A | T |

[symbol]

indicates data missing or illegible when filed

Although our current strategy depends on polygenic score from selected 6 SNPs, further validations are ongoing to design a polygenic score based on all 36 endurance SNPs.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1) A method of determining an individual's genetic predisposition to elite athletic ability, the method comprising identifying individuals with Single Nucleotide Polymorphisms (SNPs) that are in linkage disequilibrium with a reference SNP.

2) The method of claim 1, wherein said reference SNP comprises an SNP on the MYBPC3 gene.

3) The method of claim 2, wherein said reference SNP comprises rs1052373.

4) The method of claim 3, further comprising determining whether the individual or subject with SNP rs1052373 is a GG homozygote.

5) The method of claim 3, further comprising assessing VO₂ₘₐₓ in individuals positive for SNP rs1052373.

6) The method of claim 5, further comprising assessing endurance athletic ability in individuals positive for SNP rs1052373.

7) The method of claim 5, further comprising determining whether individuals with SNP rs1052373 are carriers of either of the AA+AG alleles.

8) The method of claim 5, further comprising measuring testosterone levels of individuals with SNP rs1052373.

9) The method of claim 3, further comprising measuring the level of the testosterone precursor, androstenediol (3beta, 17beta) disulfate in individuals with SNP rs1052373.

10) The method of claim 3, further comprising evaluating the training program of an individual with SNP rs1052373.

11) The method of claim 1, wherein said reference SNP comprises an SNP on the NR1H3 gene.

12) The method of claim 11, wherein said reference SNP comprises rs7120118.

13) The method of claim 12, further comprising assessing VO₂ₘₐₓ in individuals positive for SNP r rs7120118.

14) The method of claim 12, further comprising assessing endurance athletic ability in individuals positive for SNP rs7120118.

15) The method of claim 12, further comprising measuring testosterone levels of individuals with SNP rs7120118.

16) The method of claim 10, further comprising measuring the level of the testosterone precursor, androstenediol (3beta, 17beta) disulfate in individuals with SNP rs7120118.

17) The method of claim 10, further comprising evaluating the training program of an individual with SNP rs7120118.

18) The method of claim 1, wherein said elite athletic ability comprises at least one of elite endurance sport ability, elite strength sport ability, or elite speed sport ability.

19) The method of claim 1, wherein said elite athletic ability comprises elite sports ability, wherein said elite sports ability comprises an event with a low, moderate or high aerobic (dynamic) component.

20) The method of claim 1, wherein said elite athletic ability comprises elite sports ability, wherein said elite sports ability comprises an event with a low, moderate or high power component.

21) The method of claim 1, further comprising tailoring a training regime to match the predisposition for elite athletic ability.

22) An SNP chip comprising at least one of SNPs rs6455978, rs10036834, rs2292434, rs2477838, and s4824047.

23) An SNP chip comprising at least two of SNPs rs6455978, rs10036834, rs2292434, rs2477838, and s4824047.

24) An SNP chip comprising at least three of SNPs rs6455978, rs10036834, rs2292434, rs2477838, and s4824047.

25) An SNP chip comprising at least four of SNPs rs6455978, rs10036834, rs2292434, rs2477838, and s4824047.

26) An SNP chip comprising SNPs rs6455978, rs10036834, rs2292434, rs2477838, and s4824047.

27) A method of identifying elite endurance athletes with 90% efficiency comprising calculating the polygenic score of the athlete based on the presence of SNPs rs1052373, rs6455978, rs10036834, rs2292434, rs2477838, and s4824047, wherein said athlete is an elite endurance athlete if said polygenic score is greater than 0.56.
